(19) United States
(12) Plant Patent Application Publication
(10) Pub. No.: US 2013/0125270 P1
(43) Pub. Date: May 16, 2013

(54) Apple tree named 'PremA92'

(50) ;

(71) Applicant: Allan White, Havelock North (NZ)

(72) Inventor: Allan White, Havelock North (NZ)

(21) Appl. No.: 13/573,005

(22) Filed: Aug. 9, 2012

Related U.S. Application Data
(60) Provisional application No. 61/574,967, filed on Aug. 11, 2011.

(51) Int. Cl. A01H 5/00

(52) U.S. Cl. PLT/168

(57) ABSTRACT

A new and distinct apple tree named ‘PremA92’ is disclosed. The new apple is notable for its attractive appearance, superb texture, improved flavor and long shelf life.

### CROSS-REFERENCE TO RELATED APPLICATIONS

This is a Non-Provisional Plant Patent Application based upon Provisional Patent Application No. 61/574,967 and claims priority under 35 U.S.C. 199(e).

### STATEMENT REGARDING FEDERALLY SPONSORED RESEARCH OR DEVELOPMENT

None.

### LATIN NAME OF THE GENUS AND SPECIES OF THE PLANT CLAIMED

Mallus domestica Mill

### VARIETY DENOMINATION

‘PREMA92’

### BRIEF DESCRIPTION OF THE PHOTOGRAPHS

FIG. 1 shows the fruit of a ‘PremA92’ apple tree;

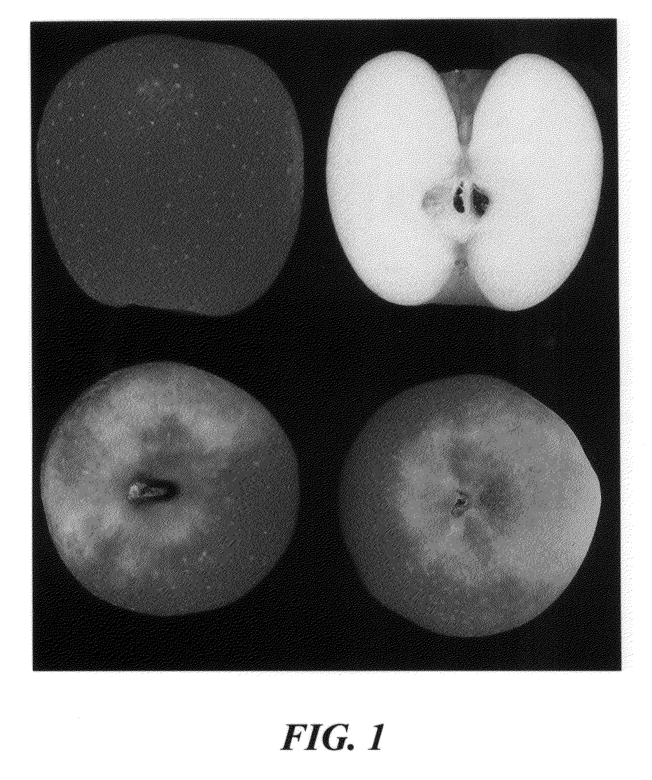

FIG. 2 shows a ‘PremA92’ apple tree;

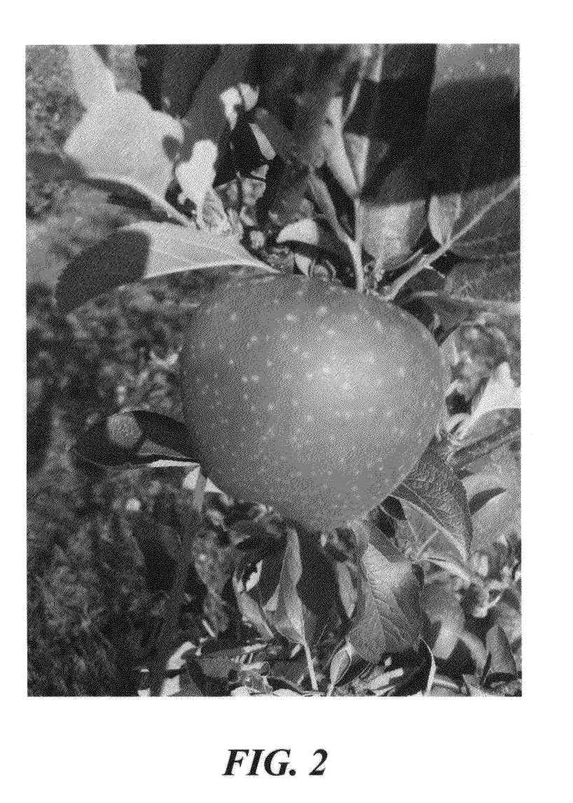

### BRIEF DESCRIPTION OF THE VARIETY

The new variety of apple tree ‘PremA92’ was created in the course of a planned breeding program carried out at The New Zealand Institute for Plant and Food Research, Hawkes Bay, New Zealand. ‘PremA92’ originated as a result of a controlled cross of ‘Fuji’ (seed parent, not patented) and ‘Sciros’ (pollen parent, U.S. Plant Pat. No. 7,814, patent expired). ‘PremA92’ was selected as a single plant from among a population of seedlings derived from the parents. Asexual propagation by budding was first carried out in New Zealand. The variety has since been observed over a number of asexually propagated generations, and has been found to remain true to type.

### DETAILED BOTANICAL DESCRIPTION OF THE VARIETY

The following detailed botanical description is based on observations made during the 2011 growing season at New Zealand. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree.—Type — Ramified; Habit — Spreading.
Fruit.—Shape — Globose; Relative area of over color — Medium; Hue of over color — Red; Pattern of over color — Only solid flush.
Time of beginning of flowering.—Medium.
Time of eating maturity.—Very late.
Comparison chart:

| Comparison | | |
|---|---|---|
| Characteristics | Fuji | T92 |
| Maturity | Late | Very late |
| Stem basin | Medium | Deep |
| Calyx basin | Medium | Deep |
| Over color | Red/Orange | Red |
| Lenticels | Not prominent | Pronounced |

Use: Fresh Market.

1. We claim a new and distinct apple tree substantially as shown and described herein.
